(19) United States
(12) Patent Application Publication
(10) Pub. No.: US 2019/0106670 A1
(43) Pub. Date: Apr. 11, 2019

(54) DEVICES, SYSTEMS, AND METHODS FOR MICROBIAL INCUBATION

(71) Applicant: SELUX DIAGNOSTICS, INC., Charlestown, MA (US)

(72) Inventors: Aleksandar Vacic, Charlestown, MA (US); Nathan Purmort, Charlestown, MA (US); Frederick P. Floyd, JR., Charlestown, MA (US); Joshua Dean, Charlestown, MA (US); George Lysy, Charlestown, MA (US)

(21) Appl. No.: 16/152,816

(22) Filed: Oct. 5, 2018

Related U.S. Application Data
(60) Provisional application No. 62/569,281, filed on Oct. 6, 2017.

(51) Int. Cl. C12M 1/00; H05K 7/14; C12M 3/00; C12M 3/06; C12M 1/02; C12M 1/32

(52) U.S. Cl. CPC C12M 41/14; H05K 7/1427; C12M 23/12; C12M 27/16; C12M 41/22; C12M 23/44

(57) ABSTRACT

The present disclosure is related to in vitro diagnostic devices, systems, and methods, particularly microbiological diagnostic devices. The systems and methods described herein can relate to automating incubation of samples including heating, agitation, automated loading and unloading, and considerations for limiting evaporation.

### PRIORITY

This application claims the benefit of priority under 35 USC § 119 to U.S. Provisional Patent Application Ser. No. 62/569,281, filed Oct. 6, 2017, which is incorporated by reference herein in its entirety and for all purposes.

### FIELD

The present disclosure is related to in vitro diagnostic devices, systems, and methods, particularly microbiological diagnostic devices. The systems and methods described herein can relate to automating incubation of samples including heating, agitation, automated loading and unloading, and considerations for limiting evaporation.

### BACKGROUND

Antimicrobials have transformed the practice of medicine, making once lethal infections more easily treatable and saving millions of lives. Quick administration of antimicrobials has been proven to reduce mortality, especially in severe infections such as septicemia. In these severe cases, highly potent, broad-spectrum antimicrobials are most often used since information about organism (e.g., species) is typically not known. These broad-spectrum antimicrobials can have serious side effects, cause organ damage, prolong recovery and hospital stays, and in some cases increase mortality. Furthermore, the overuse of antimicrobials has caused the rise of antimicrobial resistant organisms, which have become a serious and growing threat to public health. A growing body of evidence suggests that, by using targeted antimicrobial therapy, patient mortality can be reduced (e.g., minimized), recovery can be shortened, and hospitals can save money on both patient stay and minimizing usage of expensive antimicrobials.

However, complete information typically needed for targeted antimicrobial therapy is typically delivered 2-3 days after a sample is taken. Current antimicrobial susceptibility tests (AST) may require more than 8 hours to determine and deliver relevant and useful information, which is typically not sufficient to provide a same day result. Because many clinical laboratories operate on 12-hour shifts, this means that actionable AST information is not available to prescribers until the following day.

Some systems perform phenotypic AST testing of patient samples by exposing them to a set of antimicrobial dilution series and measuring their growth over time. Growth can be measured indirectly and most frequently optically by measuring solution turbidity or fluorescence of a dye triggered by microorganism metabolism. By quantitative comparison of optical signal, these systems determine the lowest concentration in dilution series of each antimicrobial that successfully inhibits growth of the tested microorganism. This value, known as minimum inhibitory concentration (MIC), is often used by clinicians to determine the most effective antimicrobial and dosage, i.e., deliver targeted antimicrobial therapy. In addition, a qualitative susceptibility result (QSR) of susceptible (S), intermediate (I) or resistant (R) may be reported with or instead of MIC.

To arrive at MIC and QSR results, the growth of a given microorganism in standardized nutrient broth (e.g., Muller Hinton broth) is compared to its growth in multiple antimicrobial dilution conditions (e.g., across a 2× dilution series). Manually, growth is typically measured only once, after 16-24 hours, as defined by Clinical & Laboratory Standards Institute (CLSI). Some automated systems, as previously mentioned, shorten this time by interrogating microorganism growth in each test well periodically (e.g., 20 minutes). This process can be tedious and is typically not performed by technicians. Growth curves are then analyzed using proprietary algorithms that include analysis of absolute, relative values between wells, rates, integrals, etc., of growth curves.

Historically, automation in microbiology clinical laboratories has lagged compared to clinical chemistry and hematology areas where automation and new assay development have reduced time from sample to result. Three major commercial automated AST systems have been developed in the past 30 years; all were designed to automate operation typically done by highly trained technicians. These automated systems perform operations that are superficially similar to the operations performed by automated systems developed in the fields of immunoassay, nucleic acid testing, cytology, etc. (e.g., sample incubation, fluid handling, and so on). However, phenotypic AST applications require these operations to be performed under conditions that are often not compatible with the design limitations of existing automated devices. These include a need for rapid agitation of microbial samples during incubation, maintenance of even heating across AST sample cartridges, and the maintenance of fluid volumes during the incubation.

One important difference between existing automated systems and AST optimized systems is in the heating of samples during incubation. Current systems generally rely on forced air heating systems to heat samples during incubation. These systems may be susceptible to the formation of temperature gradients across AST sample cartridges, and to evaporative loss of aqueous incubation media over the course of 6-8 hour incubation periods.

### SUMMARY

This disclosure addresses the limitations of existing automated AST systems by providing novel, high-performance systems and methods for AST sample incubation, which utilize, variously, incubation enclosures and/or calibrated conductive heating. The systems and methods described herein can have several advantages over conventional systems, such as those described above utilizing forced air heating systems. For example, conventional systems having forced air heating can make automated loading and unloading more complex and may require load locks and additional actuators. The increased design complexity can decrease reliability and increase cost and size of systems which, in turn, typically makes them more difficult for integration with other subassemblies, such as fluid handlers, liquid dispensers, other robotic gantries and arms, centrifuges, optical readers, etc. In addition, combining capabilities such as agitation increases the overall cost and complexity of the system. Forced air heating is also typically less uniform than conductive heating due to convective currents, particularly when doors are opened and closed and microbial sample cartridges are inserted into and removed from an incubator. However, less efficient, larger, and poorer performing forced air heating systems have been generally acceptable in conventional incubation systems because most conventional systems are designed to incubate for long periods wherein fluctuations in temperature or other inefficiencies have less of an impact. However, the devices, systems, and methods described herein, which may provide for more efficient, customized, and better controlled heating can be particularly useful in testing systems that perform faster assays in which efficient heating may play a critical role in performance.

In various embodiments of the present disclosure, an in vitro microbial incubation system may include a plurality of enclosures each having a plurality of walls, a floor, a ceiling, and an access door. Each enclosure may be configured to removably house one of a plurality of cartridges containing microbial samples. A plurality of resistive heaters may each have a heat diffuser substantially aligned with an underside of one of the plurality of cartridges. Each heater may be configured to thermally conduct heat substantially evenly across each of the plurality of cartridges. A printed circuit board (PCB) may be disposed on the incubation system across the plurality of enclosures. The PCB may be electrically coupled to each of the plurality of resistive heaters. The PCB may be configured to independently adjust a temperature across each of the plurality of cartridges. The PCB may be configured to calibrate each of the plurality of resistive heaters and may store a resulting calibration data on the PCB. An agitator may be configured to support and agitate the plurality of enclosures. The agitator may include a first stage configured to support the plurality of enclosures and may translate in a first direction. A second stage may be configured to support the first stage and may translate in a second direction substantially perpendicular to the first direction. The cartridges may be configured to be reversibly removed from the enclosures by an automated system. The access door of each of the plurality of enclosures may be configured to at least partially enclose each cartridge within each enclosure in a stable configuration and may be configured to allow reversibly removable access to each cartridge in an engaged configuration. The cartridges may include a plurality of wells in a two-dimensional array. The temperature in a first direction of the array or a second direction of the array may not vary more than 1° C. A thermal conductivity of each diffuser plate may be greater than a thermal conductivity of each enclosure.

In various embodiments, an in vitro microbial incubation system may include a plurality of enclosures. Each enclosure may have a plurality of walls, a floor, a ceiling, and an access door. Each enclosure may be configured to removably house one of a plurality of cartridges containing microbial samples. A plurality of resistive heaters may each be disposed on the floor of each of the plurality of enclosures. A plurality of heat diffusers may each be in substantial contact with each resistive heater. A PCB may be disposed on the incubation system across the plurality of enclosures and may be electrically coupled to each of the plurality of resistive heaters and may be configured to independently adjust a temperature across each of the plurality of cartridges. The plurality of cartridges may each have an underside substantially aligned with one of the plurality of heat diffusers. Each door of the plurality of enclosures may be configured to at least partially enclose each cartridge in a stable configuration. Each door may be configured to allow reversibly removable access to each cartridge in an engaged configuration. Each door may be hung at a first end above each floor such that a second end of each door swings toward a corresponding cartridge in the engaged configuration and the second end may be magnetically held in place in the stable configuration. Each door may include at least one rounded tab extending normal to an outside surface of each door and may be configured to engage an arm of an automated grabbing device. An agitator may be configured to support and agitate the plurality of enclosures. The agitator may include a first stage configured to support the plurality of enclosures and translate in a first direction. A second stage may be configured to support the first stage and may translate in a second direction perpendicular to the first direction. Each heat diffuser may substantially hold a respective cartridge stable with respect to the plurality of enclosures. The cartridges may include a plurality of wells in a two-dimensional array. The temperature in a first direction of the array or a second direction perpendicular to the first direction of the array may not vary more than 1° C. A thermal conductivity of each diffuser plate may be greater than a thermal conductivity of each enclosure. Each of the resistive heaters and diffusers may be larger than each of an array of wells of each cartridge.

In various embodiments, a method of in vitro microbial incubating may include loading a plurality of cartridges containing samples into a plurality of enclosures. Each of the plurality of cartridges may be thermally conducted substantially evenly across each of the cartridges using a plurality of resistive heaters and a plurality of heat diffusers. The plurality of enclosures may be agitated. A temperature of each of the plurality of cartridges may be independently adjusted with a controller mounted on the plurality of enclosures. One or more of the cartridges may be automatically unloaded from the plurality of enclosures at a termination of an incubation cycle. Independently adjusting the temperature may be performed by a PCB containing calibration data of the plurality of cartridges. Independently adjusting the temperature may be performed using a proportional-integral-derivative controller.

### BRIEF DESCRIPTION OF THE DRAWINGS

FIG. 1 illustrates an in vitro microbial incubation system, in accordance with an embodiment of the present disclosure.

FIGS. 2A and 2B illustrate cartridges containing microbial samples within an in vitro microbial incubation system, in accordance with an embodiment of the present disclosure.

FIG. 3 illustrates access doors of enclosures, in accordance with an embodiment of the present disclosure.

FIG. 4 illustrates a PCB, in accordance with an embodiment of the present disclosure.

FIG. 5 illustrates a temperature map across a heater of an incubation system, in accordance with an embodiment of the present disclosure.

FIGS. 6A-6C illustrate an agitator, in accordance with an embodiment of the present disclosure.

FIGS. 7A and 7B illustrate a test panel positioned over a heater and a heat diffuser, in accordance with an embodiment of the present disclosure.

FIG. 8 illustrates a block diagram of a PCB, in accordance with an embodiment of the present disclosure.

### DETAILED DESCRIPTION

### Overview

One group of embodiments described herein relates to incubation systems for use in automated AST systems. Incubation systems according to these embodiments are generally characterized by (a) one or more spaces for incubating AST test panels under conditions that favor the maintenance of consistent temperature and vapor pressure over time, and (b) the ability to hold AST test panels in place during agitation at speeds from 100 to up to 600 rotations per minute (RPM).

Test panels utilized in the various embodiments of this disclosure typically comprise, e.g., a 96 or 384 well plate or a similar vessel, as described at ¶[0067] of US pre-grant publication no. 2018/0088141 to Vacic, et al. (“Vacic 2018”), which is incorporated by reference for all purposes herein. In use, the test panels are generally uncovered to permit access by other systems of an automated AST system. For example, uncovered test panels are readily accessible to fluid-handling systems as described at ¶¶[0095]-[0100] of Vacic under the heading “fluid handling systems.”

Even when covered, test panels may be susceptible to fluid spillage and to evaporation. Spillage may result in cross-contamination of wells, and both spillage and evaporation may reduce the potential growth of cultured microbes; any of these outcomes may confound AST results, and it is desirable to avoid them.

Incubators of this disclosure reduce the potential for evaporative fluid loss from the test panels by maintaining a more consistent vapor pressure in the atmosphere surrounding each test panel compared to traditional convective heating. This is achieved, in some instances, by the use of baffles or walls which limit airflow in the area around each test panel. The baffles or walls, which may partially or fully enclose the AST test panels, define an air mass around the test panels that maintains consistent vapor pressure and temperature during an incubation period compared to the airflow involved with convective heating.

Another incubator feature that contributes to the maintenance of a consistent temperature is the use of a heating element positioned in proximity to at least a portion of the test panel. The heating element may operate by conduction or radiation, as discussed in greater detail below.

To hold the test panel in place during agitation, the incubator may incorporate one or more retentive features that connect the test panel to the incubator by means, e.g., of a mechanical fit, a magnetic fit, or an adhesive fit. AST test panels, like 96- or 384-well plates more generally, may include extended sidewalls defining a flanged bottom, one or more sockets, or other features that may be utilized to secure the test panel in an apparatus. Thus, in one set of embodiments, the incubator includes a platform having one or more recessed ends that mates with a flanged bottom of a test panel, or one or more pins that fit within one or more sockets of the test panel. Test panel retention features are discussed in greater detail below.

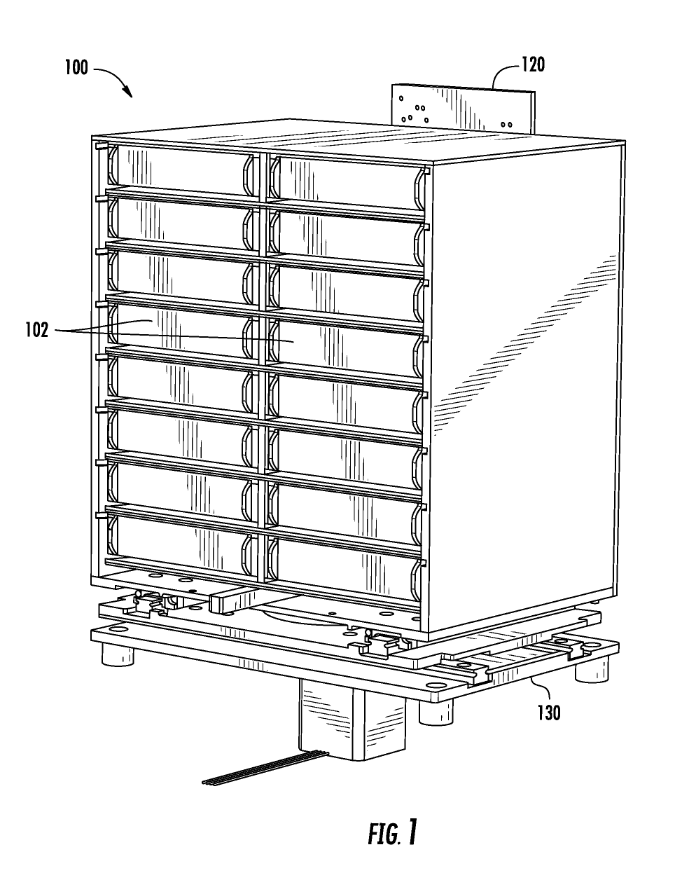

Turning to specific embodiments of the incubators of this disclosure, FIG. 1 depicts one embodiment of an in vitro microbial incubation system 100, which includes enclosures 102 configured to removably house cartridges containing microbial samples and to limit airflow and thermal exchange around the cartridges during incubation. In the depicted embodiment, heating of the enclosures 102, as reflected in a temperature of the cartridges, is controlled by a PCB 120 that communicates with one or more heating elements disposed within each enclosure 102. In embodiments described herein, PCB may include the printed circuit board and one or more components mounted thereon. The figure also depicts an agitator 130 supporting the enclosures 102, which agitator 130 is operable to agitate the enclosures 102 and cartridges of samples housed there within.

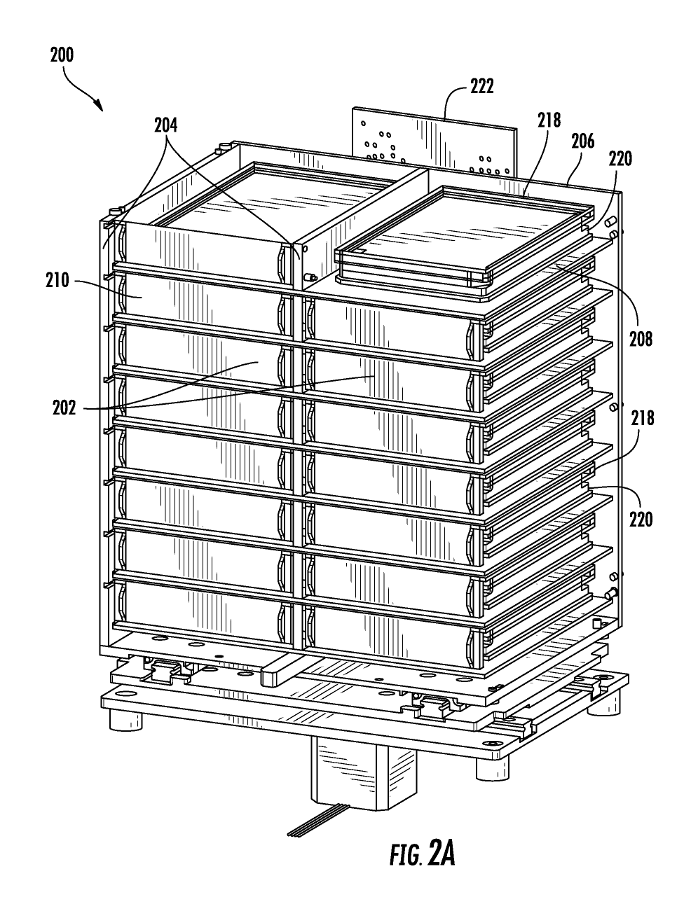

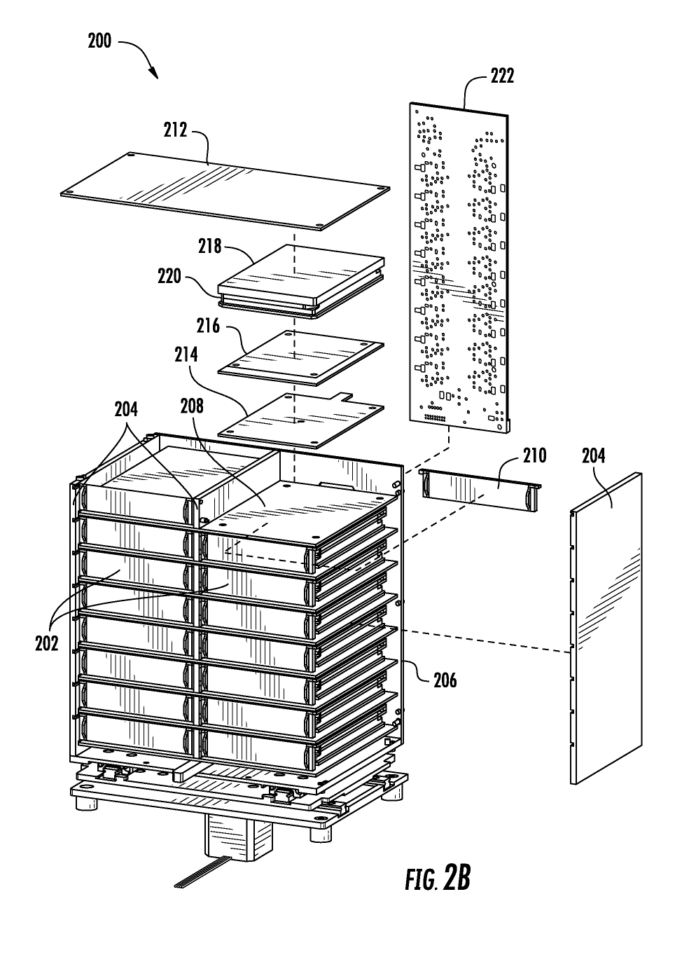

Referring to FIGS. 2A and 2B, an embodiment of an in vitro microbial incubation system 200 according to the present disclosure is illustrated with portions of the system removed and/or in an exploded view, which includes enclosures 202. Each enclosure 202 is made up of two walls 204, a back 206, a floor 208, a ceiling 212, and an access door 210. A floor 208 of one enclosure 202 acts as the ceiling 212 of another enclosure 202 and vice versa. A heater 214 (e.g., a resistive heater or other heating element) is attached to the floor 208 of each enclosure 202. A heat diffuser 216 is attached to the top of the heater 214 by, e.g., adhesive, screws, or the like. A cartridge 220 of microbial samples including a cover 218 is placed over the heater 214 and heat diffuser 216. As illustrated in FIG. 2A, the cartridge 220 completely covers the heater 214 and/or the diffuser 216. The heater 214 and/or diffuser 216 are shaped to include sides that engage the underside features of a cartridge 220 such that the cartridge 220 is substantially held in place (e.g., during agitation). The heaters 214 are controlled by a PCB 222 that is mounted on the back 206 of the system 200. The outside walls 204 and back 206 are removable, while the inner walls 204 are fastened to and/or through the floors 208. As such, the incubation system 200 is modular, allowing for the addition or removal of enclosures 202, and allowing for alternative locations for access doors 210.

In various embodiments described here or otherwise within the scope of the present disclosure, the surface(s) of each floor, heater, and/or diffuser may be structured so that they mate or otherwise interface with the bottom, underside, and/or inner surface(s) of a test panel (e.g., a cartridge or microwell plate) containing one or more samples being incubated. A heated surface (e.g., the floor, the heater, or the diffuser) in direct contact with the sample receptacle may allow for more efficient and uniform heat transfer and temperature distribution across the sample receptacle than with other heating conventions such as thermal convection. In various embodiments, each heating element may be sized to be larger than the array of wells of the sample receptacle and/or the diffuser. Oversizing the heating element such that it has a footprint that exceeds the footprint of the array of wells of the sample receptacle may reduce (e.g., eliminate) potential edge effects (e.g., uneven heating) that might otherwise occur when the heating element is only about the size of the receptacle (or smaller). In various embodiments, the cartridges may be retained in their respective enclosures by one or more mechanical retainers. The mechanical retainer may include one or more simple fixed short thin walls (e.g., flanges) between or about which the cartridge is placed by a user and/or robotic arm. In some embodiments, the mechanical retainers may define a cradle in which the cartridge sits. Additionally, or alternatively to fixed cartridge retainers, the retainers may also be active (e.g., using electronic actuators) or passive (e.g., using springs) “clamping” mechanisms to tightly constrain the sample receptacle. In some embodiments, the sample receptacle can also be constrained (or otherwise held in position) using a floor that includes a platform on which a skirted sample receptacle (e.g., microtiter plate having side portions that extend down vertically beyond other portions of the plate) is placed and utilizes the skirt as the retainer around the platform. In various embodiments, multiple cartridges may be housed within each enclosure by expanding the footprint of each enclosure in one or more lateral dimensions while still permitting human and robot access from common faces.

In various embodiments, the enclosures may include various types of heating elements, which may be disposed in one or more surfaces of the enclosure (e.g., top, bottom, and/or sides. For example, the heating elements may be resistive or semiconducting (e.g., Peltier heaters). Semiconducting elements may also be configured for cooling. In some embodiments, liquid heating or cooling may also be used by circulating a coolant through machined or cast channels in the incubator floors and then through a heat exchanger. In various embodiments, cooling may be useful for chilling enclosures and/or samples to common laboratory temperatures such as −80 C, −20 C or +4 C. Applications where cooling is useful or preferred may include chemical or reagent storage or storage of biological samples. Additionally, in various embodiments, heating or cooling elements may be included in unevenly heated regions of each enclosure that may improve uniformity. A heater may be adhered directly to a diffuser and/or the floor for efficient transfer of heat without significant interaction of air between the surfaces. A conductive diffuser allows for heat to more easily transfer from the heater and through the diffuser to the cartridge than to the surrounding enclosure (i.e., a path of least resistance for heat transfer). Such a setup allows for, e.g., maximizing heat transferring to the sample wells through the diffuser as opposed to being soaked up by the floor of an enclosure.

In various embodiments, adjacent walls, doors, floors, and backs may comprise materials that insulate the enclosure from ambient temperatures. These materials may include polymers, ceramics, metals, or the like. This insulation may assist in maintaining a consistent temperature about each panel which, in turn, may increase the consistency across the panel. The thermal isolation of each individual enclosure allows for discreet temperatures to be set in each enclosure that may be independent from other enclosures.

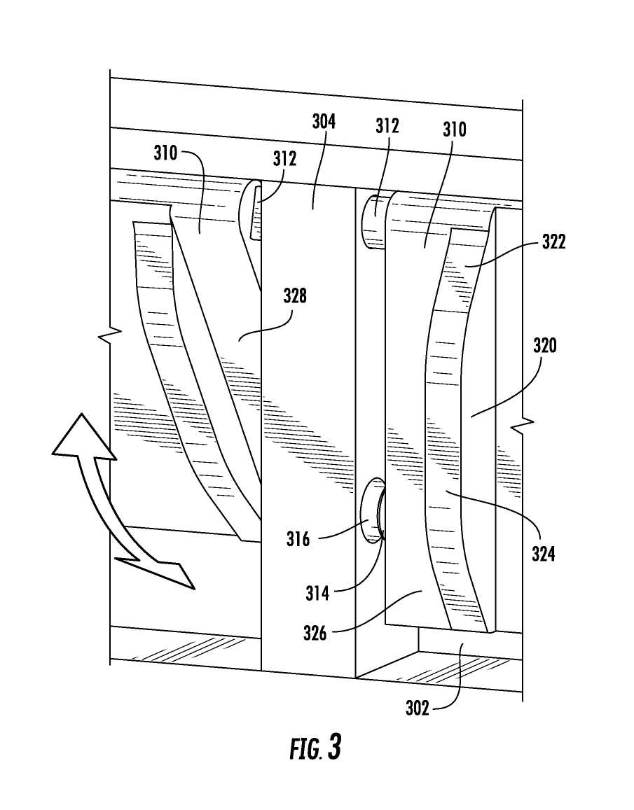

Referring to FIG. 3, an embodiment of an in vitro microbial incubation system according to the present disclosure is illustrated, which includes access doors 310 at the front of the enclosures 302. The access doors 310 are hung via an extension member 312 extending into adjacent walls 304 such that the extension member is free to rotate within the walls 304 or such that the doors 310 are free to rotate about a fixed extension member 312. The access doors 310 include one or more magnetic pins 314 at a lower end of the door 310 when the door 310 is hung by the extension member 312. The magnetic pin 314 magnetically interacts within a second magnetic pin 316 embedded within the wall 304 when the door 310 is in a closed stable configuration 326. The magnetic field between the pins 314, 316 is strong enough such that smaller forces (e.g., shaking of the enclosure 302) will not disrupt the magnetic attraction between the pins 314, 316 such that the door 310 stays in the stable configuration 326. However, the magnetic field between the pins 314, 316 is weak enough such that larger forces (e.g., deliberate robotic arm or user interaction with the door 310) can disrupt the magnetic attraction between the pins 314, 316 such that the door 310 transitions into an engaged configuration 328 by swinging about an axis of the extension member 312. The access door 310 of each of the plurality of enclosures 302 is configured to at least partially enclose each cartridge within each enclosure in the stable configuration 326 and is configured to allow supply or removable access to each cartridge in the engaged configuration 328. Two rounded tabs 320 extend from and normal to the front face of each door 310. Each tab has a profile that transitions from the door 310 at one end, to a sloped transition portion 322, to a substantially flat portion 324 in the middle, to another downward sloping transition portion 322, and to the door 310 at the other end of the tab 320. The tabs 320 allow for a robotic arm (e.g., extensions, fingers, a gripper, or the like) to engage a door 310 and transition the door 310 from the stable configuration 326 to the engaged configuration 328 by applying a force on the tabs 320 that is in a direction toward the inside of the enclosure 302. The tab 320 “rocks” along the front and/or top of the robotic arm as the robotic arm opens the door 310 into the engaged configuration 328 such that the transition portions 322 and flat portion 324 engage and slide along the robotic arm. The tabs 320 allow for the robotic arm to interact with the doors 310 without traumatizing (e.g., scratching, denting, or the like) the front face of the doors 310. The access doors 310 are rigid panels (e.g., plastic, metal, or the like), but may be flexible membranes (e.g., rubber, or the like) pre-slit to permit passage of the sample receptacle and still provide an adequate thermal barrier for the enclosure 302.

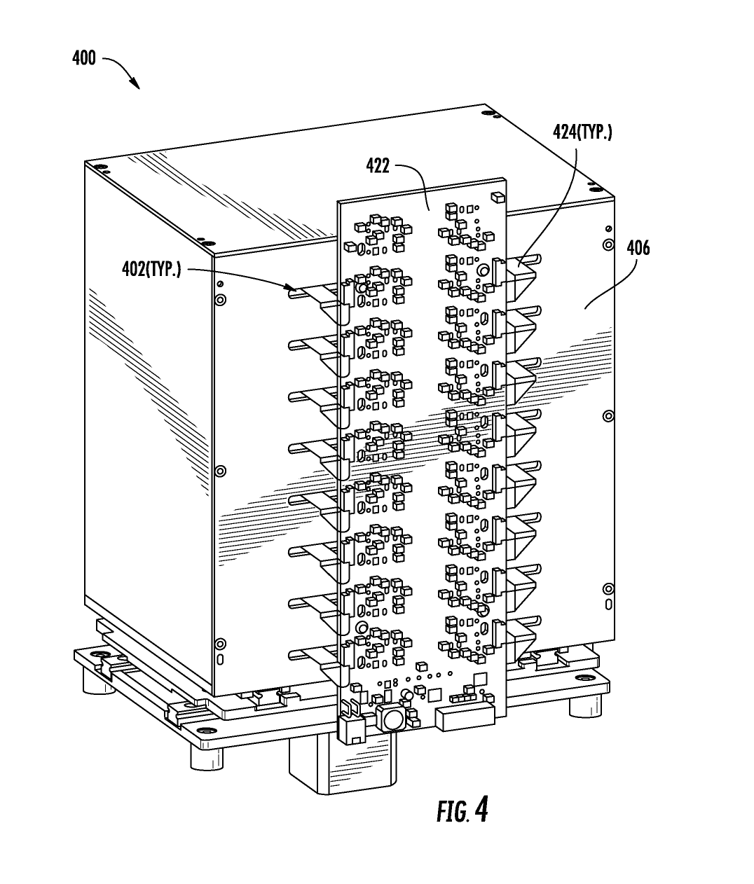

Referring to FIG. 4, an embodiment of an in vitro microbial incubation system 400 according to the present disclosure is illustrated, which includes a PCB 422 mounted onto the back 406 of the incubation system 400 across the plurality of enclosures 402. Because the PCB 422 is mounted directly to the back 406, it may easily travel with the system 400 and does not need to be anchored, connected, or monitored separately from the system 400. The PCB 422 is electrically coupled to each of the heating elements within each enclosure 402 via connectors 424. The connectors 424 are flexible and extend from the heaters to the PCB through the back of each enclosure 402. The PCB 422 is configured to independently control the thermal activity of each heating element within each enclosure. In this way, the PCB 422 is able to individually monitor and adjust a temperature across each of the plurality of cartridges.

In various embodiments described here or otherwise within the scope of the present disclosure, each enclosure may include one or more temperature sensing elements (e.g., a thermocouple, thermistor, resistance temperature detectors (RTD), p-n junction, etc.) and may be configured to allow for individual (e.g., customized, optimized, tailored, varied per sample, etc.) adjustment of heating temperatures. For example, a PCB may have a separate RTD channel for each enclosure (e.g., 16 RTD channels). Separate RTD channels for each enclosure allows each RTD chip to be located near the flexible connector leading to each enclosure rather than a central RTD microprocessor with connections throughout the PCB leading to the enclosures that may be impeded by noise emitting from other components on the PCB. Such temperature sensing elements may be included within the enclosure, within the heating element, within the heat diffuser, and/or within the cartridge. In various embodiments, a system may use an active feedback controller (e.g., using proportional-integral-derivative (PID) control, bang-bang control, or other control scheme). The controller (whether integrated per enclosure or separate) may include any of various connection interfaces, such as a simple communication interface (e.g., 1-wire or 2-wire (e.g., inter-integrated circuit (I2C), integrated inter-IC sound bus (I2S), system management bus (SMBUS))). Devices for thermal feedback and control may be arranged in any of various implementations. In some embodiments, the density of thermal feedback sensors may be customized (e.g., optimized) for a variety of design goals. For low-cost applications, a single thermal sensing element may be placed within each enclosure and a simple bang-bang style controller may be used to maintain relatively consistent (e.g., constant) temperatures for that enclosure and/or cartridge. For applications where acute accuracy is desired or required, a high density (e.g., 8, 16, 96, 384, 1536, or the like) array of thermal sensors may be arranged on, within, or near the cartridge, heating element, diffuser, and/or enclosure. This array of sensors may be arranged to correspond with critical areas of the cartridge. For high-accuracy applications, sophisticated PID style controllers and/or programmed microcontrollers may be used to vary the amount of current delivered to the heating/cooling elements to maintain a consistent (e.g., constant) temperature across the sensor matrix of the array.

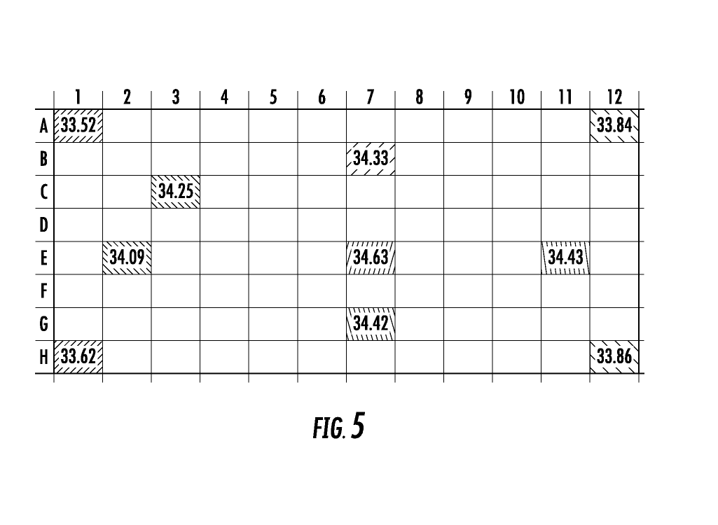

Referring to FIG. 5, a heat map of a heater embodiment of an in vitro microbial incubation system according to the present disclosure is illustrated. This map is laid out in a grid formation with an x-axis numbered 1 through 12 and a y-axis labeled A through H to identify and describe the position of a temperature sensor across a heater. In this embodiment, there are sensors at locations A1, A12, B7, C3, etc. Sensors are located at key locations such as the perimeter of the heater (i.e., A1, A12, H1, and H12) where it may be expected that the heater is the coolest, and at the general center of the heater (i.e., E7) where it may be expected that the heater is the warmest. Such sensors, although in the heater, are in intimate contact with a diffuser, which is in close proximity with the wells of a test panel. As illustrated, the sensors across this heater do not vary from the set temperature of 35° C. by more than 0.5° C. Exemplary heaters may be controlled to not vary from a target temperature by more than a tolerance of 0.5° C. Exemplary target temperatures for each heater may be, e.g., about 35° C., about 35.5° C., about 34° C. to about 37° C., about 33° C. to about 38° C., etc.

In various embodiments, a substantially uniform temperature across each cartridge of an incubation system may be obtained. Such uniform temperature may be monitored by the PCB via one or more sensors in an array across each cartridge. Such arrays may be located within the enclosure, the heating element, the heat diffuser, and/or the cartridge. The PCB may read the temperature of each array and may have a calibration transfer function for each resistive element that monitors and records a history of temperature data of the array. Such temperature calibration data may be stored on the PCB for a controller to ramp up to and maintain a desired temperature of the array. Such stored data may also be logged and reported for an incubation session such that a user may examine the temperature of each cartridge during the session. Such data does not need to be bussed out to a processor outside of the incubation system, as the PCB may process, record, and report the data to a user within the incubation system itself. The wells of the cartridges may be arranged in a two-dimensional array. The PCB may control the resistive heaters such that the temperature of the array of wells in a first direction (e.g., an x-axis) of the array or a second perpendicular direction (e.g., a y-axis) of the array does not vary more than 1° C. For example, a cartridge of wells may be held at a temperature of about 35° C. with each well not varying from that temperature by more than about 0.5° C.

In some embodiments, a cartridge of an array of sample wells may include outside rows and/or columns of wells that are cooler than inner wells due to uneven distribution of heat across the array of wells and uneven loss of heat across the wells. This phenomenon may affect samples where microbial growth is sensitive to temperature. Therefore, it may be advantageous to include microbial samples in these cooler wells that are not sensitive to temperature. Otherwise, measured results (e.g., growth or lack of growth) from these cooler sample wells may vary from other wells or a control well not because of their microbial contents, but because of the temperature gradient across the wells. Such results, e.g., growth or a lack of growth, may not be attributable to, e.g., an antimicrobial, because the catalyst for such a result may instead be the difference in temperature. Even with a controlled tolerance of temperatures across the wells, the coolest wells of a cartridge may sit on the outside border of an array of wells. Such temperature gradients across an array of wells may need to be considered when designing a test protocol such that the temperature gradient does not impact the measured results of an incubation cycle. This may include leaving outside wells of an array empty, filled with fluid(s) not associated with the sample batch to be incubated, or filled with another batch of samples not sensitive to temperature.

In various embodiments, resistive heaters may reduce undesirable evaporation of samples during incubation. The thermal conduction of heat transfer provided by resistive heaters may be desirable over convection heating because the enclosures will not succumb to translation of air that may vary the humidity of each enclosure and may interact with the atmosphere in contact with the microbial samples. Convectional heating may promote evaporation and may undesirably disrupt the samples within the cartridges. The effects of evaporation on microbial growth may be particularly significant in test panels comprising large numbers of wells, e.g., 96 wells, 384 wells, etc., which may comprise small fluid volumes (e.g., less than 200 μl of fluid per well) and/or relatively high surface area to volume ratios. Incubators of this disclosure which implement conductive heating systems will be less prone to evaporation, generally, than incubators utilizing convectional heating.

Embodiments of an incubator assembly may be mounted directly or indirectly to an agitator configured for continuous or partial agitation (e.g., shaking) during an incubation session. An agitator may include one or more devices to generate one or more linear, orbital, and/or semi-orbital motions with continuous or periodically defined duty cycles. In various embodiments, force, speed, and displacement may be adjustable to allow specific agitation performance (e.g., receptacles, such as cartridges with more sample wells (e.g., 384 well micro titer plates)) that may require a smaller orbital radius compared to cartridges with fewer sample wells (e.g., 96 well plates).

In various embodiments, the incubator assembly may be mounted to one or more stages that may drive the assembly in a circular, semi-circular, ellipsoid, axial or bi-axial motion that allows agitation of receptacles loaded into the assembly. In some cases, orbital speed can be adjustable. In some embodiments, radius or displacement of the motion can also be variable.

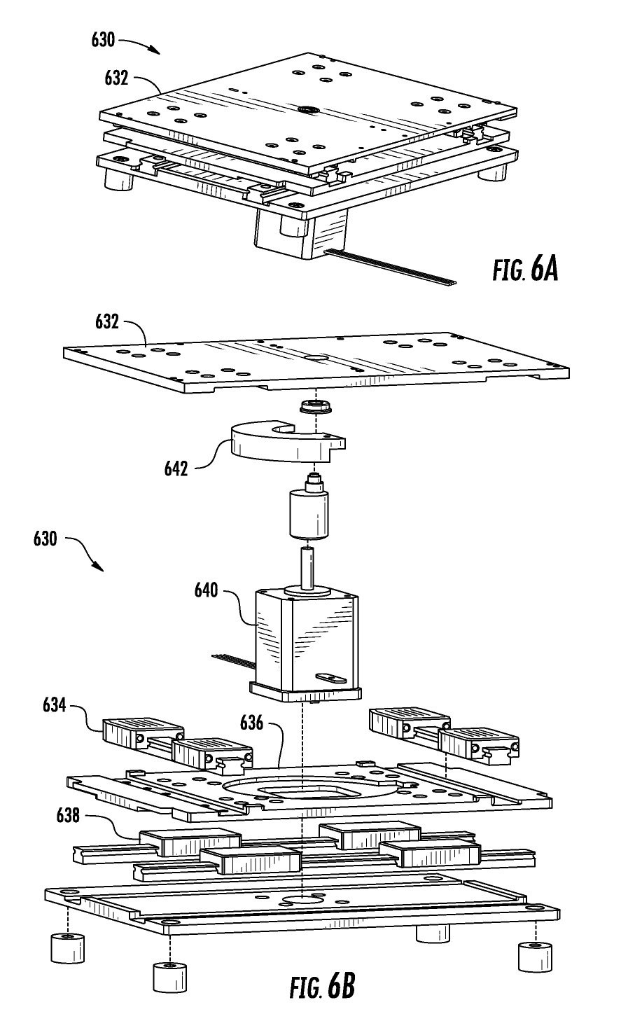

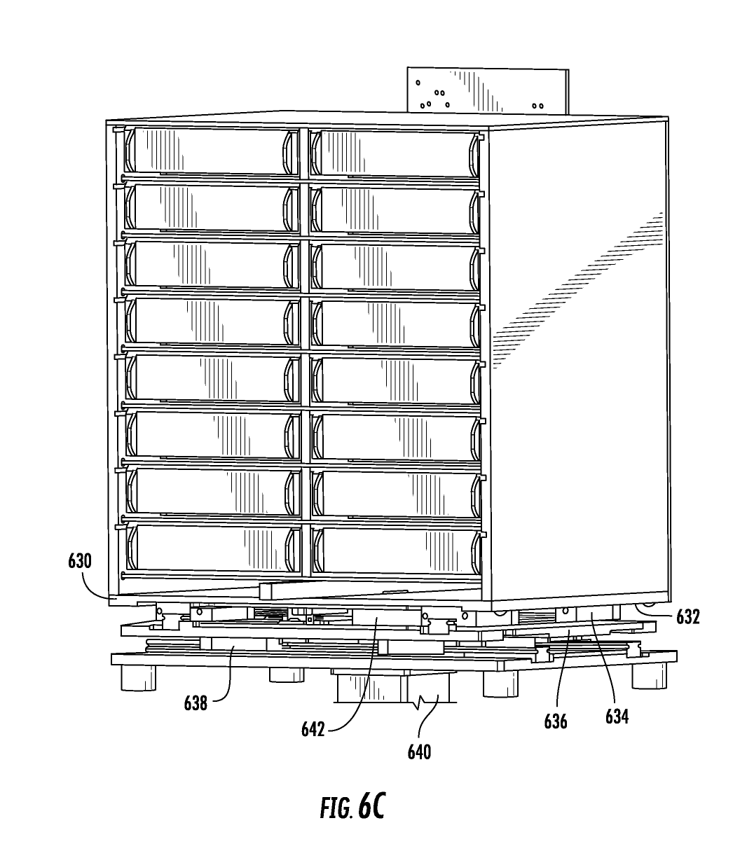

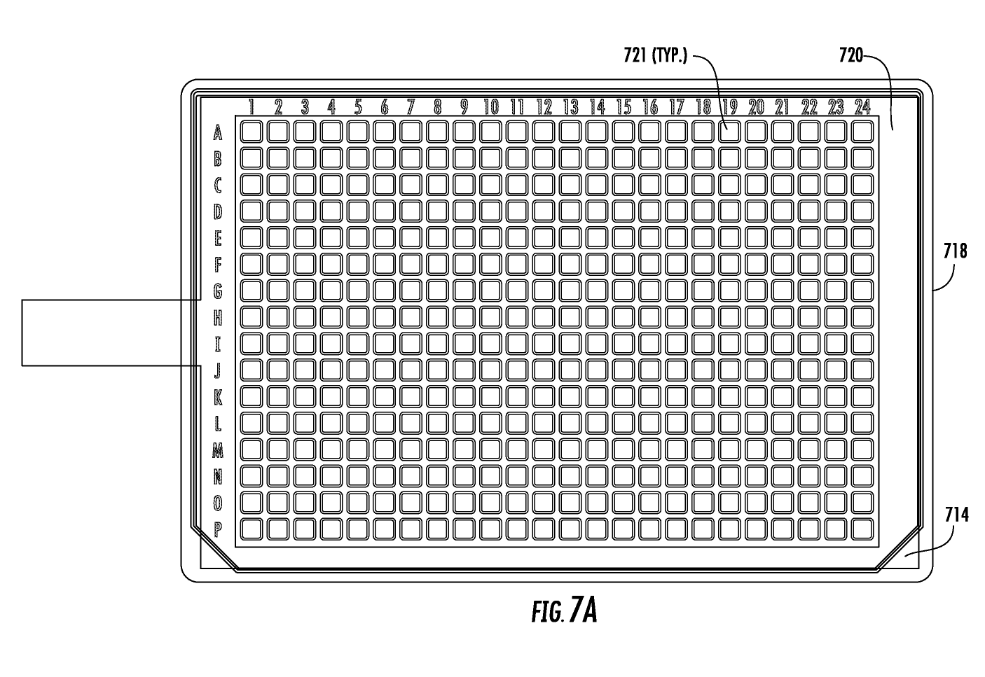

Embodiments of an incubator may introduce agitation to provide for better (e.g., more rapid and steady) growth during incubation when compared to stationary (i.e., unagitated) incubation and can be achieved by agitating the sample(s) in a manner that enables oxygenation and better distribution of growth media nutrients throughout a cartridge well. Any of various agitation systems can be implemented to impart motion on the samples. For example, in various embodiments, referring to FIGS. 6A-6C, an agitation driver system 630 includes a primary stage 632 configured to support numerous enclosures. The primary stage 632 is supported by a set of linear bearings 634 that allow the primary stage 632 to translate in a first direction (e.g., in one orthogonal direction, along an x-axis, or the like). The linear bearings 634 of the primary stage 632 are supported by a secondary stage 636. The secondary stage 636 is supported by another set of linear bearings 638 that allow the secondary stage to translate in a second direction that is substantially perpendicular to the first direction (e.g., in a normal orthogonal direction, along a y- axis, or the like). Combined, the primary stage 632 allowing for translation in the first direction, and the secondary stage 636 allowing for translation in the second direction normal to the first direction permit the numerous enclosures to move in two-dimensions while being agitated. The agitation system utilizes a combination of rotational and linear motion to generate sample shaking. The combined linear motions of the stages 632, 636 along different axes can be used to generate a substantially orbital motion on the enclosures and cartridges therein.

A motor 640 is actuated to impart an orbital motion to an off-center weight 642 about a drive shaft of the motor 640 to shake the enclosures in a substantially circular motion. In various embodiments, a controller of the motor 640 controls the orbital speed and radius of the motion of a stage 632, 636 by varying the orbital speed and radius of the weight 642 to achieve a variety of different agitations for the enclosures.

In various embodiments, the agitation subsystems may be used in association with the incubation subsystems or as stand-alone subassemblies. The agitation subsystem may include a motor (e.g., a servo, or the like) that spins a rotor having an off-center (e.g., eccentric, or the like) interface, such as a cam device, that imparts a rotational, oscillating motion onto the enclosures when the motor spins a drive shaft in accordance with the various agitation methods described herein. In some cases, the drive shaft and/or a rotor may include a counter balance weight to reduce or limit vibration during agitation. In various embodiments, the one or more stages may be disposed along one or more bearing surfaces (e.g., linear bears, rollers, or the like) to provide smooth and undisrupted translation along the stage(s)'s oscillating path. Additional bearings may be used per stage for added stability.

An agitation embodiment may include a variable orbital speed and/or radius during operation. For example, a radius of the orbital agitation (e.g., orbital radius) of the sample can be less than about 25 mm (e.g., about 1 mm to about 12 mm, about 1 mm to about 10 mm, about 1 mm to about 8 mm, about 1 mm to about 3 mm, about 2 mm to about 3 mm, about 6 mm, or the like). The driver of an agitation system may be driven by any of various combinations of motors, belts, gears, cams, and/or other electromechanical components. In some cases, orbital speed and radius of motion can be user adjustable and adjusted (e.g., optimized) for different panel formats and samples to be tested. For example, 384-well plates can be agitated along an orbit having a diameter of about 4 millimeters and 96-well plates can be agitated along an orbit having a diameter of about 8 millimeters.

In addition to orbit diameter, orbital rotation speed can also affect microorganism growth rates. For example, the orbital shaking may occur at a frequency of greater than about 50 revolutions per minute. In some examples, the orbital shaking may occur at a frequency of greater than about 350 revolutions per minute. In some examples, the orbital shaking may occur at a frequency of less than about 750 revolutions per minute. In some examples, the orbital shaking may occur at a frequency of about 150 revolutions per minute. For example, speeds between about 150 revolutions per minute and about 650 revolutions per minute may promote acceptable rates of microorganism growth. In some cases, it may not be necessary for the agitation of the cartridges to be performed continuously throughout the incubation time, but a duty cycle of at least 10% may be beneficial.

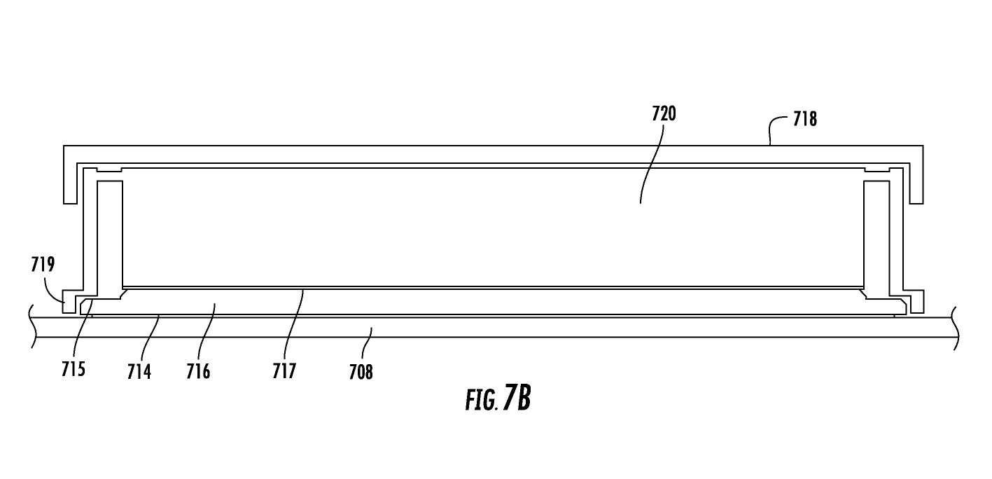

Referring to FIGS. 7A and 7B, an embodiment of an in vitro microbial incubation system according to the present disclosure is illustrated, which includes a test panel 720 positioned over a heater 714 and a heat diffuser 716. The cartridge 720 is resting on a floor 708 of an enclosure and includes a transparent cover 718 on top. The heater 714 and the diffuser 716 have a larger two-dimensional footprint (i.e., length and width) than the footprint of the array of wells 721. The diffuser 716 includes an engagement portion 717 in intimate contact with the cartridge 720 at—and extending larger than—the array of wells 721. The diffuser also includes a border portion 715 having a height less than that of the engagement portion 717. The border portion 715 extends about the diffuser 716 and is adjacent to the foundation 719 of the cartridge 720 so that the border portion 715 prevents significant movement of the cartridge 720 by interfacing with the foundation 719 should the cartridge be agitated. Other exemplary features of the diffuser and/or cartridge may include, e.g., the inclusion of recessed edges or proud shoulders that mate with the flanged underside of a cartridge. Such intimate contact may allow for a more consistent temperature across a test panel than without such contact. The close proximity of a diffuser to a liquid analyte minimizes the time needed for that liquid to reach a desired temperature. For example, with ambient temperatures ranging from 16° C. to 32° C., a panel will reach 35° C. in less than 18 minutes. A diffuser may comprise various materials such as copper, aluminum, aluminum alloys, a combination of these, or the like. In various embodiments, the heater can be designed to allow for higher resistive heating at the perimeter of the said heater. This compensates heat losses along the edges. Such heaters and/or diffusers may be used not only beneath a cartridge, but about the cartridge in other orientations such as, e.g., above and adjacently.

In various embodiments, a method of in vitro microbial incubating may include loading a plurality of cartridges containing samples into a plurality of enclosures. Each of the plurality of cartridges may be thermally conducted substantially evenly across each of the cartridges using a plurality of resistive heaters and a plurality of heat diffusers. The plurality of enclosures may be agitated. A temperature of each of the plurality of cartridges may be independently adjusted with a controller mounted on the plurality of enclosures. One or more of the cartridges may be automatically unloaded from the plurality of enclosures at a termination of an incubation cycle. Independently adjusting the temperature may be performed by a PCB containing calibration data of the plurality of cartridges. Independently adjusting the temperature may be performed using a proportional-integral-derivative controller.

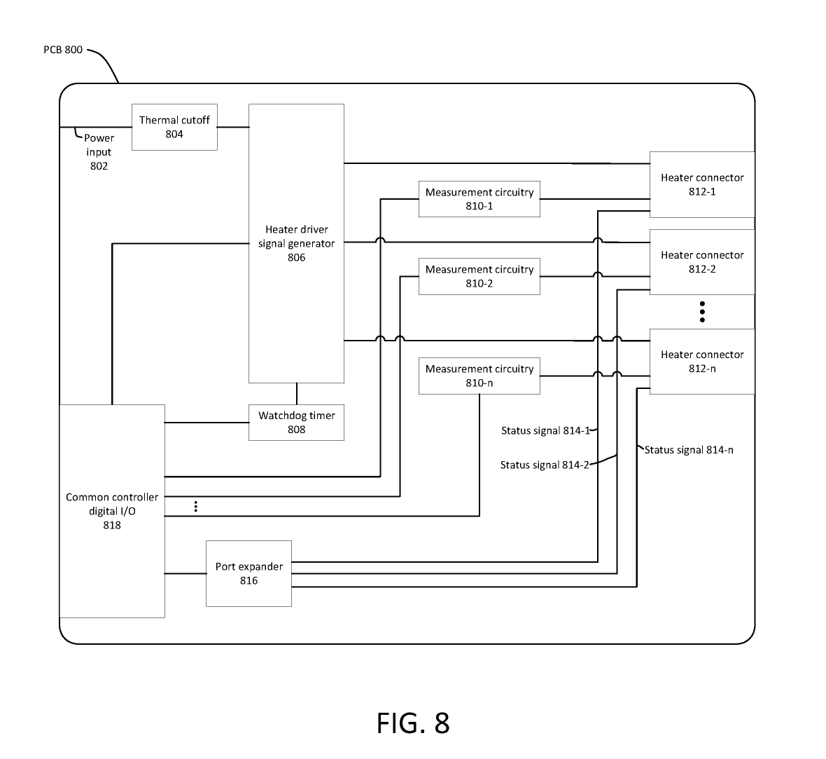

Referring to FIG. 8, a block diagram of a PCB 800 according to the present disclosure is illustrated. In some embodiments, PCB 800 and/or components thereof may be the same or similar to one or more other PCBs and/or component thereof described herein (e.g., PCBs 120, 222, 422). In the illustrated embodiment, PCB 800 may include a power input 802, a thermal cutoff 804, a heater driver signal generator 806, a watchdog timer 808, one or more measurement circuits 810-1, 810-2, 810-n, one or more heater connectors 812-1, 812-2, 812-n, a port expander 816 for receiving one or more status signals 814-1, 814-2, 814-n from one or more heaters, and a common controller digital input/output (I/O) 818. In various embodiments, the components of PCB 800 may operate to enable one or more functional aspects of one or more heaters or corresponding sensors described herein. For example, PCB 800 may include electrical circuitry and components for controlling and monitoring one or more heaters (e.g., heater 214) connected thereto. Embodiments are not limited in this context.

In many embodiments, each of one or more heaters may be connected to PCB 800 via a respective one of heater connectors 812-1, 812-2, 812-n (or heater connectors 812). In many such embodiments, the heater connectors 812 can enable various signals to be sent to and/or received from a connected heater. In many such embodiments, these signals may include one or more of a drive signal, a measurement signal, and a status signal. For example, heater driver signal generator 806 may provide a drive signal to a heater coupled to heater connector 812-1, measurement circuitry 810-1 may exchange measurement signals with the heater coupled to heater connector 812-1, and common controller digital I/O 818 may receive status signals from the heater coupled to heater connector 812-1 via port expander 816. In some embodiments, these signals may be communicated via serial peripheral interfaces (SPIs). In some such embodiments, the SPIs may perform analog-to-digital and/or digital-to-analog conversions. It will be appreciated that for ease of description various features may be described utilizing measurement circuitry 810-1 and heater connector 812-1, however these features may apply equally to the other measurement circuitry and heater connectors. Further, signals communicated via a common heater connector and/or signals communicated via different heater connectors may be independently controlled.

As will be described in more detail below, common controller digital I/O 818 may generally direct and/or manage operation of PCB 800. Further, common controller digital I/O 818 may enable PCB 800 to interface with external components other than the heaters. For instance, a target temperature for an enclosure may be received by interfacing with a user interface. In some such instances, common controller digital I/O 818 may implement one or more operations or procedures to achieve the target temperature in the enclosure. In some embodiments, common controller digital I/O 818 may include one or more PID controllers. In some such embodiments, a PID controller may enable a feedback loop for heater control using the corresponding measurement circuitry and heater driver signal generator 806. In various embodiments common controller digital I/O 818 may include processing circuitry and a memory. In various such embodiments, the memory may comprise instructions that when executed by the processing circuitry cause the processing circuitry to perform one or more operations or realize one or more embodiments described herein.

In the illustrated embodiment, heater driver signal generator 806 may receive power from power input 802 via thermal cutoff 804. In various embodiments, the power input 802 may provide 24 volts to heater driver signal generator 806. In some embodiments, thermal cutoff 804 may provide a safety mechanism that disconnects heater driver signal generator 806 from power input 802 if an ambient temperature exceeds a threshold. For instance, if the temperature proximate thermal cutoff 804 exceeds 75 degrees Celsius, power input 802 may be disconnected from heater driver signal generator 806. In some embodiments, thermal cutoff 804 may include a thermal fuse.

In many embodiments, heater driver signal generator 806 may independently provide drive signals to connected heaters at the direction of common controller digital I/O 818. In many such embodiments, common controller digital I/O 818 may provide characteristics of a signal, such as a pulse width modulated signal, to be generated by heater driver signal generator 806 and provided to a connected heater. For example, common controller digital I/O 818 may provide a duty cycle or voltage level for a drive signal generated by heater driver signal generator 806. In some such examples, common controller digital I/O 818 may cause heater driver signal generator 806 to provide a different duty cycle or a different voltage level to each connected heater to independently control temperatures within different enclosures (e.g., enclosures 102).

In various embodiments, watchdog timer 808 may provide a safety mechanism to prevent erroneous or dangerous operation, such as resulting from failure of the common controller digital I/O 818. In various such embodiments, if watchdog timer 808 expires it may cause heater driver signal generator to shut down. For instance, under normal operation, common controller digital I/O 818 may periodically reset watchdog timer 808. However, a failure of common controller digital I/O 818 will prevent the watchdog timer 808 from being reset, resulting in the expiration of watchdog timer 808, and causing heater driver signal generator 806 to shut down.

In some embodiments, common controller digital I/O 818 may utilize measurement circuitry to monitor a temperature proximate a connected heater. For instance, measurement circuitry 810-1 may be connected to a temperature probe, such as an RTD, included in a heater. In such instance, measurement circuitry 810-1 may report the temperature proximate to the heater to common controller digital I/O 818 either periodically or at the request of the common controller digital I/O 818. In one or more embodiments, common controller digital I/O 818 may receive a status signal from each connected heater via port expander 816. In one or more such embodiments, port expander 816 may include an SPI expander. In other embodiments, common controller digital I/O 818 may receive status signals for each connected heater without use of a port expander. In one or more such embodiments, the status signal may indicate whether a corresponding heater is functioning properly.

Various embodiments and/or components of PCB 800 may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor. Some embodiments may be implemented, for example, using a machine-readable medium or article which may store an instruction or a set of instructions that, if executed by a machine, may cause the machine to perform a method and/or operation in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non- removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low- level, object-oriented, visual, compiled and/or interpreted programming language. Conclusion

This disclosure has focused on a handful of discrete embodiments, with the intention of illustrating the principles of the systems and methods described. These descriptions are intended to be illustrative rather than limiting. Unless otherwise defined, all technical terms used herein have the same meaning as commonly understood by one of ordinary skill in the art to which the disclosure belongs.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. The term “or” is generally employed in its sense including “and/or” unless the content clearly dictates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

As used herein, the terms “cartridge,” “test panel,” “panel,” “cassette,” “plate”, “microwell,” and any plural derivative of these terms are meant to be interchangeable. As such, features, uses, etc. described with reference to one or more of these terms are intended to apply to other references and embodiments of these terms unless clearly stated otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effectuate such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

Finally, while certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein are not mutually exclusive and can exist in various combinations and permutations, even if such combinations or permutations are not made expressly herein, without departing from the spirit and scope of the invention. In fact, variations, modifications, and other implementations of what is described herein will occur to those of ordinary skill in the art without departing from the spirit and the scope of the invention. As such, the invention is not to be defined only by the illustrative description herein.

What is claimed is:

1. An in vitro microbial incubation system comprising:
a plurality of enclosures each having a plurality of walls, a floor, a ceiling, and an access door, each enclosure configured to removably house one of a plurality of cartridges containing microbial samples;
a plurality of resistive heaters each having a heat diffuser substantially aligned with an underside of one of the plurality of cartridges and each heater configured to thermally conduct heat substantially evenly across each of the plurality of cartridges; and
a printed circuit board (PCB) electrically coupled to each of the plurality of resistive heaters, the PCB configured to independently adjust a temperature of each of the plurality of cartridges.

2. The system of claim 1, wherein the PCB is configured to calibrate each of the plurality of resistive heaters and store a resulting calibration data on the PCB.

3. The system of claim 1, further comprising an agitator configured to support and agitate the plurality of enclosures, the agitator comprising:
a first stage configured to support the plurality of enclosures and translate in a first direction; and
a second stage configured to support the first stage and translate in a second direction substantially perpendicular to the first direction.

4. The system of claim 1, wherein the cartridges are configured to be reversibly removed from the enclosures by an automated system.

5. The system of claim 1, the access door of each of the plurality of enclosures is configured to at least partially enclose each cartridge within each enclosure in a stable configuration and configured to allow reversibly removable access to each cartridge in an engaged configuration.

6. The system of claim 1, wherein the cartridges comprise a plurality of wells in a two- dimensional array, wherein the temperature in a first direction of the array or a second direction of the array does not vary more than 1° C.

7. The system of claim 1, wherein a thermal conductivity of each diffuser plate is greater than a thermal conductivity of each enclosure.

8. An in vitro microbial incubation system comprising:
a plurality of enclosures each having a plurality of walls, a floor, a ceiling, and an access door, each enclosure configured to removably house one of a plurality of cartridges containing microbial samples;
a plurality of resistive heaters each disposed on the floor of each of the plurality of enclosures;
a plurality of heat diffusers each in substantial contact with each resistive heater;
a printed circuit board (PCB) electrically coupled to each of the plurality of resistive heaters, the PCB configured to independently adjust a temperature of each of the plurality of cartridges; and
wherein the plurality of cartridges each have an underside substantially aligned with one of the plurality of heat diffusers.

9. The system of claim 8, wherein each door of the plurality of enclosures is configured to at least partially enclose each cartridge in a stable configuration and configured to allow reversibly removable access to each cartridge in an engaged configuration.

10. The system of claim 9, wherein each door is hung at a first end above each floor such that a second end of each door swings toward a corresponding cartridge in the engaged configuration and the second end is magnetically held in place in the stable configuration.

11. The system of claim 9, wherein each door includes at least one rounded tab extending normal to an outside surface of each door and configured to engage an arm of an automated grabbing device.

12. The system of claim 8, further comprising an agitator configured to support and agitate the plurality of enclosures, the agitator comprising:
a first stage configured to support the plurality of enclosures and translate in a first direction; and
a second stage configured to support the first stage and translate in a second direction perpendicular to the first direction.

13. The system of claim 8, wherein each heat diffuser substantially holds a respective cartridge stable with respect to the plurality of enclosures.

14. The system of claim 8, wherein the cartridges comprise a plurality of wells in a two- dimensional array, wherein the temperature in a first direction of the array or a second direction perpendicular to the first direction of the array does not vary more than 1° C.

15. The system of claim 8, wherein a thermal conductivity of each diffuser plate is greater than a thermal conductivity of each enclosure.

16. The system of claim 8, wherein each of the resistive heaters and diffusers are larger than each of an array of wells of each cartridge.

17. A method of in vitro microbial incubating comprising:
loading a plurality of cartridges containing samples into a plurality of enclosures;
thermally conducting each of the plurality of cartridges substantially evenly across each of the cartridges using a plurality of resistive heaters and a plurality of heat diffusers;
agitating the plurality of enclosures; and
independently adjusting a temperature of each of the plurality of cartridges with a controller mounted on the plurality of enclosures.

18. The method of claim 17, further comprising automatically unloading one or more of the cartridges from the plurality of enclosures at a termination of an incubation cycle.

19. The method of claim 17, wherein the independently adjusting the temperature is performed by a PCB containing calibration data of the plurality of cartridges.

20. The method of claim 17, wherein the independently adjusting the temperature is performed using a proportional-integral-derivative controller.
